(19) United States
(12) Patent Application Publication
(10) Pub. No.: US 2011/0144678 A1
(43) Pub. Date: Jun. 16, 2011

(54) Endoscopic Scissors Instrument

(76) Inventor: Charles R. Slater, Fort Lauderdale, FL (US)

(21) Appl. No.: 12/638,308

(22) Filed: Dec. 15, 2009

(51) Int. Cl. A61B 17/3201; A61B 17/94

(52) U.S. Cl. 606/170

(57) ABSTRACT

An endoscopic scissor instrument includes an elongate hollow member and first and second scissor blades each having longitudinally angled distal features. At least one scissor blade is rotatably mounted in a clevis adjacent the distal end of the hollow member. The clevis includes a pivot mechanism and spring bias means, disposed adjacent the pivot mechanism on at least one external side of the first and second scissor blades, for biasing transverse movement of the scissor blades toward one another. The spring bias means of the clevis and the angling of the distal features of the scissor blades serves to generate spring forces acting on the respective cutting edges such that there is an automatic preloading force imparted between the cutting edges of the scissors' blades.

This application claims priority from U.S. patent application Ser. No. 12/335,656 filed on Dec. 16, 2008 which is hereby incorporated by reference in its entirety.

### BACKGROUND OF THE INVENTION

1. Field of the Invention

The invention relates to surgical scissors instruments and, more particularly, to endoscopic scissor instruments having small-sized scissor blades.

2. State of the Art

Endoscopy is a minimally invasive diagnostic medical procedure that is used to assess the interior of the human body using an endoscope. An endoscope generally consists of a rigid or flexible tube, an fiber optic illumination system to guide light provided by a light source through the tube of the endoscope in order to illuminate the organ or object under inspection, and a viewing system for collecting an image of the organ or object under inspection and for recording the image on an internal CCD device (video-endoscope) or for transmitting the image through the tube via a fiber optic bundle to an external video processor for viewing (fiber-endoscope). The endoscope can include one or more “operating” channels (typically 2-4 mm in diameter) that provide for passage of specialized medical instruments through the endoscope and into the field of view. Such specialized instruments (which can include biopsy forceps, brushes, needles, snares, scissors, graspers, cutters, clip appliers, etc.) can be used to take biopsies and retrieve organs (or pieces thereof) and/or foreign objects from the inside of the body. In all flexible endoscopes the distal end (4″-8″) is remotely steerable by the operator turning knobs on the back-end of the endoscope. This enables general direction control of the scope and any accessory instrument that may be in its working channel. In some instruments (especially those with lateral-viewing optics), the distal tip of the operating channel incorporates a small deflectable elevator or bridge, which permits some directional control over the instrument exiting therefrom. These general principles apply to most endoscopes, but specific instruments differ in length, size, stiffness, as well as other characteristics as the instruments are typically designed for a particular application. Endoscopy can involve, for example, the gastrointestinal tract such as the esophagus, stomach and duodenum, small intestine, and colon. It can also involve the respiratory tract, the urinary tract, the female reproductive system, and the organs of the chest. It can also involve the interior of a joint (arthroscopy). Many endoscopic procedures are considered to be relatively painless and, at worst, associated with moderate discomfort.

Laparoscopy is a minimally invasive surgical technique in which operations in the abdomen or thorax are performed through small incisions (usually 0.5-1.5 cm) via a laparoscope. There are generally two types of laparoscopes, including a telescopic rod lens system that is usually connected to a video camera (single chip or three chip) and a digital laparoscope where the camera is placed at the end of the laparoscope, thus eliminating the rod lens system. A fiber optic cable system connected to a light source (halogen or xenon) is inserted through a surgical port to illuminate the operative field for viewing. The abdomen is usually insufflated with carbon dioxide gas to create a working and viewing space. Specialized surgical instruments can be introduced into the abdomen or thorax through a surgical port in order to take biopsies and retrieve organs (or pieces thereof) and/or foreign objects from the inside of the body.

The specialized surgical instruments used for endoscopy, laparoscopy or arthroscopy generally include end effector means (e.g., graspers, cutters, forceps, scissors, clip appliers, etc.) mounted adjacent the distal end of a tube or coil. Handles (or other actuation control means) are mounted to the proximal end of the tube or coil and move an actuator axially through the tube or coil. The distal end of the actuator is mechanically coupled to the end effector means in a manner that transforms the axial movement of the actuator into the desired movement of the end effector means. Such specialized endoscopic, laparoscopic or arthroscopic surgical instruments are collectively referred to herein as endoscopic surgical instruments or endoscopic instruments. These general principles apply to most endoscopic instruments, but specific endoscopic instruments differ in length, size, stiffness, as well as other characteristics as the instruments are typically designed for a particular application as such instruments can be used for a wide variety of minimally invasive surgical procedures, including the endoscopic, laparoscopic and arthroscopic applications summarized above.

Endoscopic surgical scissors instruments generally include a pair of scissor blades pivotably mounted adjacent the distal end of a tube or coil. The scissor blades have sharpened edges that effect cutting of tissue during pivotal movement of the scissor blades relative to one another. Handles (or other actuation control means) are mounted to the proximal end of the tube or coil and move an actuator axially through the tube or coil. The distal end of the actuator is mechanically coupled to the scissor blades in a manner that transforms the axial movement of the actuator into pivoting movement of the scissor blades.

Endoscopic scissors instruments may be generally classified as either “single acting” or “double acting.” In a single acting instrument, a stationary scissor blade is supported adjacent the distal end of the tube or coil and a movable scissor blade is coupled to the distal end of the actuator and is supported adjacent the distal end of the tube or coil for rotation relative to the stationary scissor blade in accordance with actuation transmitted by the actuator. In double acting instruments, two scissor blades are coupled to the distal end of the actuator and supported adjacent the distal end of the tube or coil for rotation relative to one another in accordance with actuation transmitted by the actuator.

The construction of the scissor blades theoretically supplies a moving contact point between the opposing cutting edges as the scissor blades are closed by their pivotable movement. In order to effect a smooth cutting action, the engaging cutting edges must be kept in a moving contact point throughout the closing of the scissor blades. Typical scissor designs usually accomplish this by the use of any of the following methods: firstly, via a rigid mechanism or feature that biases the scissor blades together as the scissor blades are closed; secondly, by dimensioning the blades with a longitudinally bowed profile that forces the opposed scissor blades against each other as the scissor blades are closed and lastly by a very accurately constructed assembly with no mechanical slop in the dimensions of, or the positioning of, the scissors' blades or related components

The rigid biasing means of the first example typically is accomplished by tightening the scissors' pivot nut to remove all dimensional slop in the assembly or with a rigid cammed surface behind the pivot area that effects biasing of the scissor blades closer together as they close over each other. In the second method, which is used most commonly for larger or longer scissor blades, such as those in a standard full-sized scissor as used in regular “open” surgery, a bowed-profile that runs along the longitudinal axis of the scissor blade forces the cutting edges together. This method gives a mostly adequate cutting performance for open style surgical scissors. However for smaller scissor blades such as those used in endoscopic devices, the total loss of resiliency, due to the stiffness of small blades, means that a bowed profile in the scissor blade will not work and will only result in the contacting cutting edges gouging each other or quickly wearing away. Therefore in the currently available endoscopic scissor devices such small non-resilient and rigid blades must be designed to maintain the edge to edge contact through the use of components with very stringent dimensional accuracies, tight tolerances and tight fits. This last design method involves difficult and costly assembly and manufacturing processes. In addition, the effects of using rigid cams or similar features of the prior art in the design of small endoscopic scissors is limited by the remoteness of the cam surface from the cutting edges and because of persistent assembly “slop” offers little improvement to the problem of maintaining edge to edge contact. These design schemes have historically failed to give small surgical scissor instruments the desired sensitive feel and cutting performance that surgeons require and are familiar with through their experience in open surgery using larger hand-held surgical scissors.

### SUMMARY OF THE INVENTION

The invention provides an endoscopic scissors instrument with small-size scissor blades with improved cutting performance through an improved biasing means whereby features contained in and as part of the blade itself automatically provide a preload to its cutting edge as two scissor blades move past one another.

In another aspect, the invention provides an endoscopic scissors with small size scissor blades with improved cutting performance through an improved biasing means whereby features of the clevis provide preload to the cutting edges as the two scissor bladed move past one another.

The invention also provides such an endoscopic scissors instrument that avoids inherently expensive components, assembly and manufacturing processes.

According to the invention, an endoscopic scissors instrument includes an elongate hollow member having a proximal end and a distal end, an actuator that moves axially through the hollow member, and first and second scissor blades with respective cutting edges. At least one of the first and second scissor blades are rotatably coupled to the hollow member adjacent its distal end. At least one of the first and second scissor blades includes a base supporting a resilient leaf-spring portion that defines a respective cutting edge. The resilient leaf-spring portion extends from the base in a cantilevered arrangement along the length of the base. The cantilevered leaf-spring arrangement and angling of the leaf-spring portion serves to generate a spring force acting on the respective cutting edge such that, when in a loaded state, there is an automatic preloading force imparted between the cutting edges of the scissors' blades that maintains a consistent and continuous mating force between the two opposed sharpened cutting edges, preferably over the complete range of rotational movement of the scissor.

In another aspect of the invention, an endoscopic scissors instrument includes an elongate hollow member having a proximal end and a distal end, an actuator that moves axially through the hollow member, and first and second scissor blades rotatably mounted in a clevis adjacent the distal end of the hollow member. The first and second scissor blades each have a respective distal feature that defines a cutting edge. The distal features of the first and second scissor blades are longitudinally angled to ensure that the cutting edges are in intersection planes as the cutting edges contact one another during rotational movement of the scissor blades relative to one another. The clevis includes a pivot mechanism and spring bias means, disposed adjacent the pivot mechanism on at least one external side of the first and second scissor blades, for biasing transverse movement of the scissor blades toward one another. The spring bias means of the clevis and the angling of the distal features of the scissor blades serves to generate spring forces acting on the respective cutting edge such that, when in a loaded state, there is an automatic preloading force imparted between the cutting edges of the scissors' blades that maintains a consistent and continuous mating force between the two opposed cutting edges, preferably over the complete range of rotational movement of the scissor.

In one embodiment, the spring bias means comprises at least one leaf spring arm that is rigidly secured to a hub proximally disposed from the pivot mechanism. The at least one leaf spring extends generally parallel the longitudinal axis of the clevis and has a thru-hole coaxially aligned with thru-holes of the first and second scissor blades for receiving the pivot mechanism. The spring bias means can further comprise a tension spring that surrounds the pivot mechanism.

In another embodiment, the spring bias means comprises at least one spring washer that is coaxially aligned with thru-holes of the first and second scissor blades and receives said pivot mechanism. In this configuration, the spring washer is disposed between a pivot support feature and one of the first and second scissor blades.

In another embodiment, the spring bias means comprises at least one leaf spring with a thru-hole that is coaxially aligned with thru-holes of the first and second scissor blades for receiving the pivot mechanism. In this configuration, the leaf spring is disposed between a pivot support feature and one of the first and second scissor blades.

It will be appreciated that the endoscopic scissor instruments of the present invention provides improved edge to edge preload of the opposed scissor blades and thus enables superior cutting quality and operator feel for endoscopic scissor instruments where historically it has not been available.

Additional advantages of the invention will become apparent to those skilled in the art upon reference to the detailed description taken in conjunction with the provided figures.

### BRIEF DESCRIPTION OF THE DRAWINGS

FIG. 1 is a side view of an exemplary endoscopic scissors instrument that embodies the present invention.

FIG. 2 is an isometric view of the distal portion of the endoscopic scissors instrument of FIG. 1 in accordance with the present invention where the scissor blades of the instrument are positioned in an open configuration.

FIG. 3 is an isometric view of the distal portion of the endoscopic scissors instrument of FIG. 1 in accordance with the present invention where the scissor blades of the instrument are positioned in a closed configuration.

FIGS. 4A and 4B are schematic views of the scissor blades of the endoscopic scissors instrument of FIGS. 1-3 in accordance with the present invention.

FIG. 5A is a side view of one of the scissor blades of FIGS. 4A and 4B in accordance with the present invention.

FIG. 5B is a cross-sectional view of the scissor blade of FIG. 5A along the section labeled 5B-5B in FIG. 5A.

FIG. 5C is a cross-sectional view of the scissor blade of FIGS. 5A and 5B along the section labeled 5C-5C in FIG. 5B.

FIGS. 6A and 6B are front cross-sectional views of the respective scissor blades of the instrument of FIGS. 1-3 along section lines similar to 5B-5B in FIG. 5A which illustrate the relief angles of the cutting features of the respective scissor blades relative to the corresponding blade supports in accordance with the present invention; the cross hatching of the section is omitted to more clearly show the relief angles depicted therein.

FIG. 6C is a cross-sectional view of the scissor blade of FIG. 6B along the section labeled 6C-6C in FIG. 6B which illustrates the blade bias angle of the cutting feature of the respective scissor blade relative to its blade supports in accordance with the present invention; the cross hatching of the section is omitted to more clearly show the blade bias angle depicted therein.

FIG. 6D is an isometric view of the distal portion of an endoscopic scissors instrument in accordance with another embodiment of the present invention.

FIG. 7A is a front perspective view of an exemplary end-effector assembly of an endoscopic scissors instrument according to the present invention.

FIGS. 7B and 7C are bottom cross-sectional views of the end-effector assembly of FIG. 7A in accordance with the present invention where the scissor blades of the instrument are positioned in an open configuration.

FIGS. 7D and 7E are bottom cross-sectional views of the end-effector assembly of FIG. 7A in accordance with the present invention where the scissor blades of the instrument are positioned in a fully-closed configuration.

FIG. 8A is a front perspective view of another exemplary end-effector assembly of an endoscopic scissors instrument according to the present invention.

FIG. 8B is a bottom cross-sectional view of the end-effector assembly of FIG. 8A in accordance with the present invention where the scissor blades of the instrument are positioned in an open configuration.

FIG. 9 is a front perspective view of yet another exemplary end-effector assembly of an endoscopic scissors instrument according to the present invention.

### DETAILED DESCRIPTION OF THE PREFERRED EMBODIMENT

For purposes herein, the “distal end” of a surgical instrument or any part thereof, is the end most distant from the surgeon and closest to the surgical site, while the “proximal end” of the instrument or any part thereof, is the end most proximate the surgeon and farthest from the surgical site.

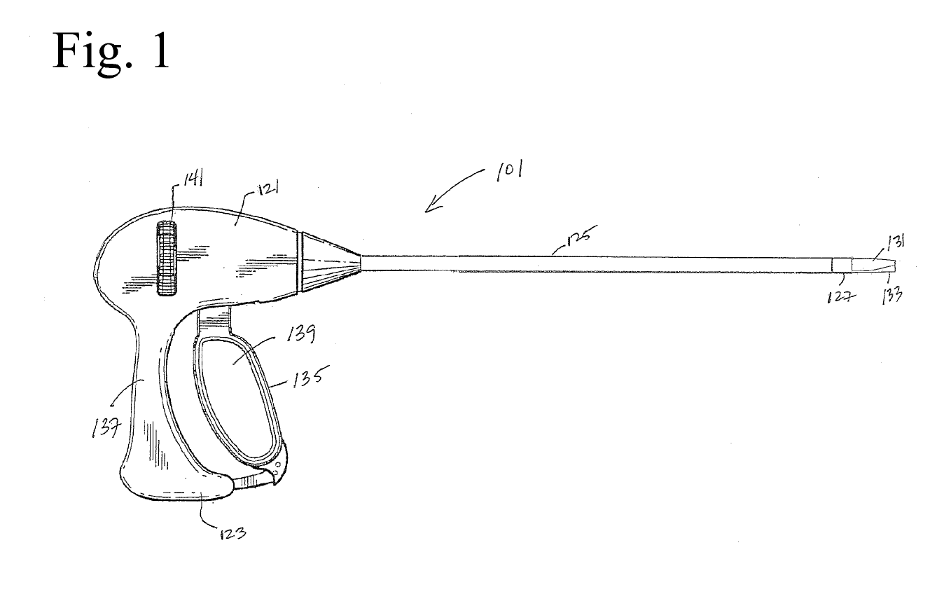

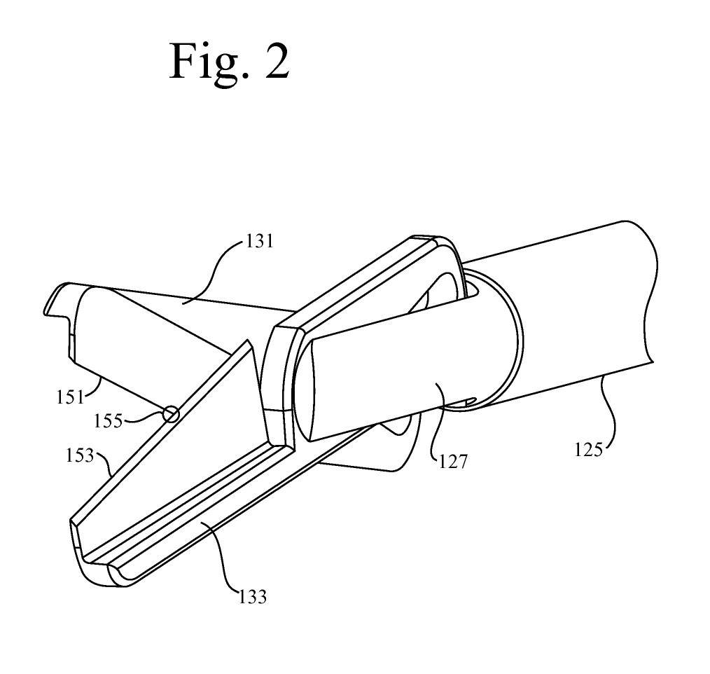

Turning now to FIGS. 1 and 2, an exemplary endoscopic scissors instrument 101 in accordance with the invention includes a housing 121 for supporting a handle assembly 123. A hollow tubular member 125 is provided with a proximal end fixably coupled to the housing 121 and a distal end fixably coupled to a clevis 127. The hollow tubular member 125 can be a coil to provide for bending and flexibility or can be a rigid or operator plastically deformable tube. A push rod actuator (not shown) extends through the hollow tubular member 125 to the clevis 127. The push rod actuator is coupled to a pair of scissor blades 131, 133 via linkages, cams or other suitable coupling features and the scissor blades 131, 133 are rotatably mounted in the clevis 127 by a pivot post (not shown). In this configuration, axial movement of the push rod actuator within the hollow tubular member 125 causes the scissor blades 131, 133 to rotate around the post and thus pivot relative to one another. Additional details of the hollow tubular member 125, the clevis 127, and the push rod actuator may be obtained by reference to U.S. Pat. No. 5,192,298 to Smith et al., herein incorporated by reference in its entirety. It will also be appreciated that other actuating mechanisms and other mechanisms for causing rotation of the scissor blades could be utilized for the endoscopic scissors instrument of the invention. Indeed, rather than using a clevis with a post around which the scissor blades rotate, the scissor blades could be provided with arcuate grooves as disclosed in U.S. Pat. No. 4,712,545 to Honkanen, herein incorporated by reference in its entirety. The invention applies to single acting and double acting endoscopic surgical scissors. It will be appreciated by those skilled in the art that other mechanisms for linking the actuation mechanism to the scissor blades 131, 133 may be utilized, such as links and pins, or a pin riding in cammed slots, or other suitable actuating mechanism. Indeed, if desired, in a single acting instrument, the push rod or actuating wire could be directly connected to the scissor blade, and in double acting instruments, two connected push rods or actuating wires could be utilized for direct connection to the scissor blades.

In the illustrative embodiment, the handle assembly 123 includes a movable front handle 135 and a fixed rear handle 137. The front handle 135 has an aperture 139 defined therethrough which enables a user to grasp and move the front handle 137 relative to the rear handle 137. More particularly, front handle 135 is selectively moveable by the user from a first position offset from the rear handle 137 to a second position in closer proximity to the rear handle 137. Such movement is transmitted to axial movement of the push rod actuator 50 extending through the hollow tubular member 125 in order to impart pivotal movement of the scissor blades 131, 133 relative to one another. A control wheel 141 can be supported within the housing 121 and extend through sidewalls of the housing 121 to allow the user to rotate together the hollow tubular member 125, the clevis 127 and the scissor blades 131, 133 mounted thereto or to rotate the clevis 127 and the scissor blades 131, 133 independently of and separately from, the hollow tubular member 125.

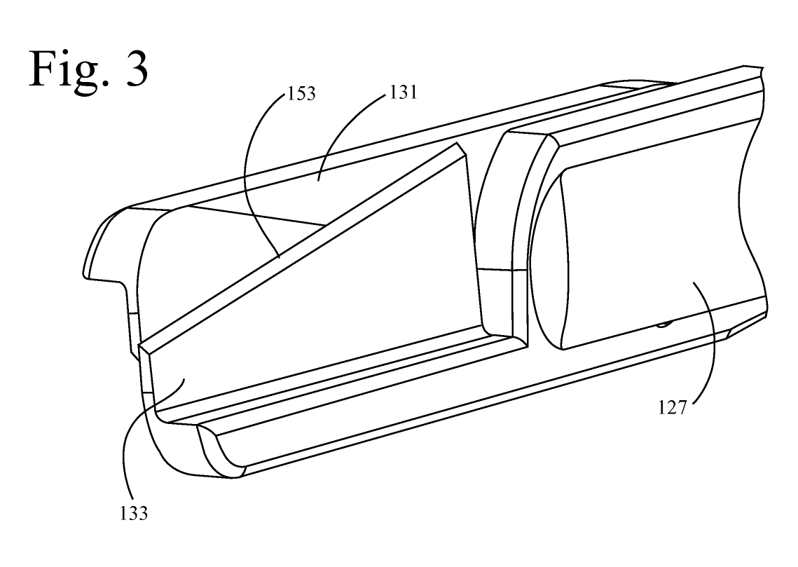

As shown in FIGS. 2 and 3, each of the scissor blades 131, 133 is provided with an inside cutting edge 151, 153 that contact one another as the scissor blades 131, 133 pivotably rotate relative to one another during use. During such rotation, a point of contact of the cutting edges 151, 153 moves along the cutting edges. In an open configuration, the point of contact is nearer to the pivot point or clevis (FIG. 2). As the blades close, the point of contact moves further from the pivot point or clevis (FIG. 3). In FIG. 2, the scissor blades 131, 133 are shown in an open configuration where the cutting edges 151, 153 are in bearing contact near the pivot point at a point shown generally by the circled portion 155.

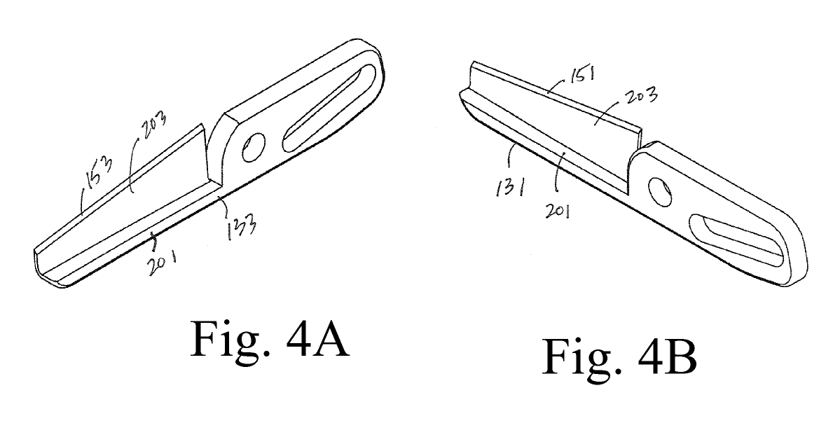

FIGS. 4A and 4B show a schematic view of scissor blades 131, 133, each of are realized by two unitary parts 201, 203. The first part 201, referred to herein as a “blade support”, is thicker and stiffer than the second part 203, referred to herein as a “cutting feature.” The thin cutting feature 203 includes a sharpened cutting edge (151,153) that extends along the entire length of the top edge of the cutting feature 203 preferably with a tapered profile as shown. Other profiled designs, such as a stepped profile or other variable profile can be used.

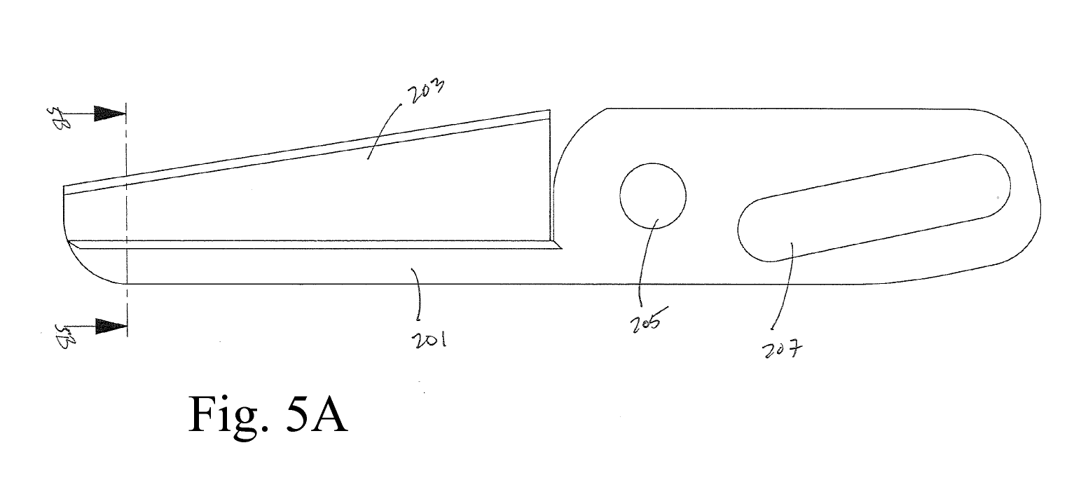

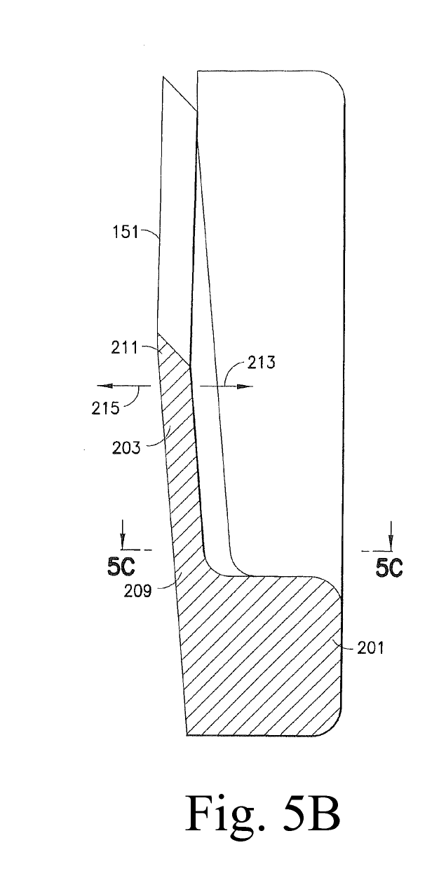

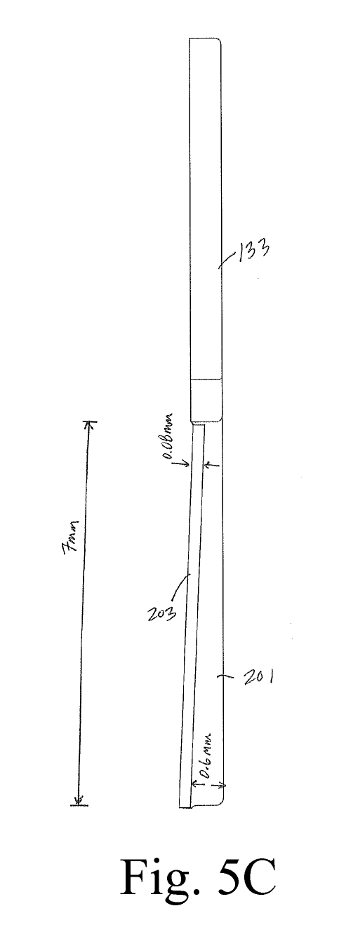

As shown in FIG. 5A, the blade support 201 includes a thru-hole 205 that receives a pivot post (not shown) as well as a cam-slot 207 disposed proximal to the thru hole 205 that receives a cam pin (not shown) connecting to the distal end of the actuator rod of the instrument. This arrangement provides for pivotal movement of the scissor blades 131, 133 relative to another in response to axial movement of the actuator rod as is well known.

As best shown in the cross-section of FIG. 5B, the thin cutting feature 203 of the scissor blades 131, 133 realizes a cantilever spring arrangement by fixing its bottom portion 209 to the blade support 201 with its top portion 211 angled or otherwise arranged to hold a bias along the length of the respective sharpened cutting edge (labeled 151 in FIG. 5B) that will ensure that the cutting edge intersects the opposing blade's cutting edge in a scissor assembly. In this cantilever spring arrangement, the thin cutting feature 203 acts as a resilient leaf-spring that allows for resilient deflection of the top portion 211 of the cutting feature 203 relative to its bottom portion 209 being rigidly held and positioned by the thick blade support 201. This allows the sharpened cutting edge 204 to forcibly engage with the opposing blade's cutting edge in a resilient and deflective manner so no gouging or wear damages the cutting edges. Such resilient deflection is depicted by vector arrow 213 in FIG. 5B. The cantilever spring arrangement of the cutting feature 203 extends along the length of the cutting feature 203 such that the resilient deflection of the top portion 211 relative to its bottom portion 209 and the blade support 201 is provided along the entire length of the cutting feature 203. The cantilever spring arrangement of the cutting feature 203 also provides a spring moment that is primarily directed across the cutting edge of the cutting feature 203 laterally outward away from the blade support 201 in the direction of vector arrow 215 as shown in FIG. 5B.

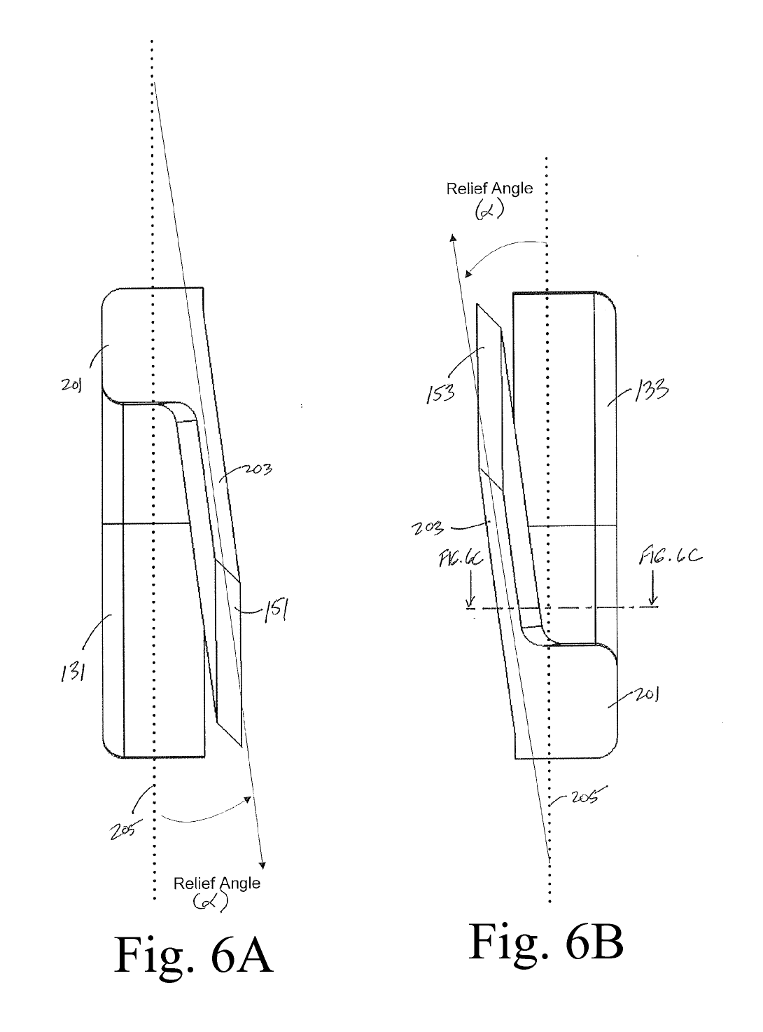

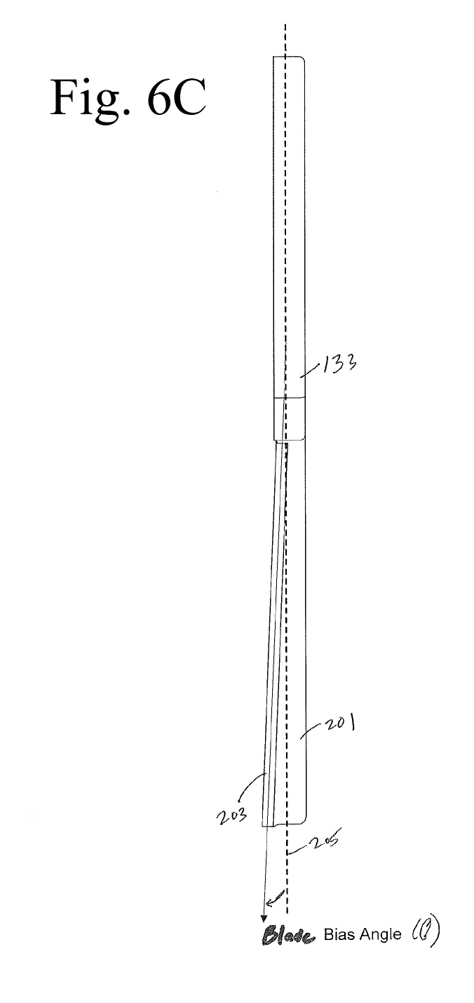

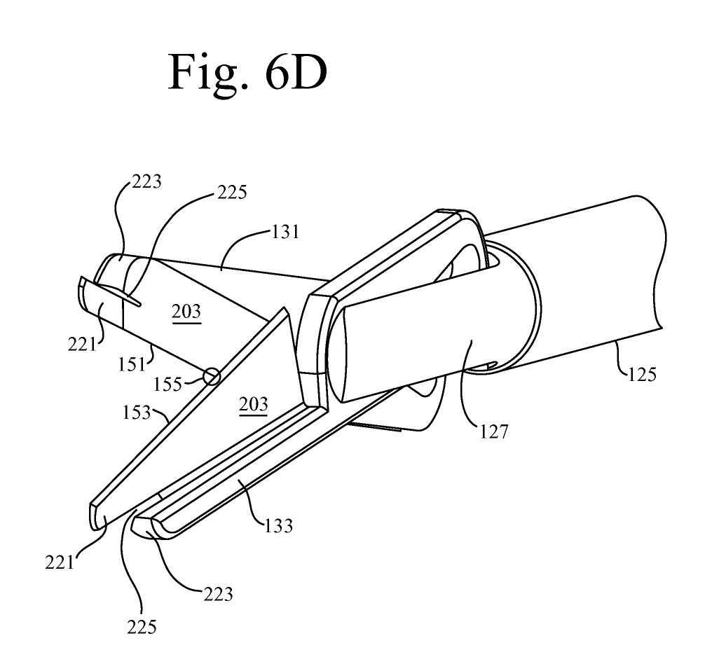

It is also contemplated that the distal portion 221 of the cutting feature 203 of the respective blades 131, 133 can extend beyond the distal end 223 of the base 201 of the respective blades as illustrated in FIG. 6D. Moreover, distal portions of the cutting feature 203 can be supported above the base 201 with space 225 provided between the base 201 and the cutting feature portion 203 to provide voids therebetween as shown. Similarly, void space can be disposed between intermediate portions and/or proximal portions of the cutting feature 203 and base 201. These features provide for greater flexibility of the cutting feature 203 in a desired location.

The cantilever spring arrangement and positional bias of the cutting features 203 ensure that the cutting edges 151, 153 of the two blades 131, 133 are in intersecting planes as the blades 131, 133 are closed. In the preferred embodiment as illustrated in FIGS. 6A-6C, the opposed cutting features 203 extend from respective base supports 201 at a relief angle α relative to the rotational planes 205 of the respective scissor blades. Moreover, as best shown in FIG. 6C, the lengthwise profile of the respective cutting features 203 of the scissor blades are angled at a blade bias angle β relative to the rotational planes 205 of the scissor blades. The bias angle of the cutting features of the two blades point toward one another as is evident from FIGS. 6A and 6B. In an illustrative embodiment, the relief angle α of the cutting features is in the range between 3° and 7° (more preferably on the order of 5°) and the blade bias angle β of the cutting features is in the range between 0.5° and 3° (more preferably on the order of 1.5°). Importantly, the relief angle α and the blade bias angle β of the cutting features 203 are provided such that selectively only the cutting edges 151, 153 of the two blades 131, 133 are on intersecting planes and therefore edge to edge contact one another is insured as the blades 131, 133 are closed. These design aspects of the leaf-spring provide a necessary blade-to-blade preload force as the blades 131, 133 are closed, which maintains a consistent and continuous forceful contact of the two opposed cutting edges 151, 153 over the complete range of rotational movement of the scissor blades 131, 133. Using this design strategy enables a small scissor to use components and manufacturing techniques with much lower quality standards without need of the high tolerance and ultra fine positioning that is presently required in surgical scissors while elevating the cutting ability and feel to a level beyond that of existing endoscopic and other small surgical scissors.

In the preferred embodiment, the blade support 201 of the respective blade has a thickness between 0.25 mm and 5 mm, while the cutting feature 203 of the respective blade has a thickness between 0.05 mm and 0.5 mm and a length less than 50 mm and preferably a the range between 5 mm and 20 mm. FIG. 5C illustrates an exemplary embodiment where the blade support 201 has a maximal thickness of 0.6 mm, and the cutting feature 203 has a thickness of 0.08 mm and a length of 7 mm. In the preferred embodiment, the scissor blades 131, 135 (including the cutting features 203 of the respective blades) are realized from high tensile strength stainless steel such as high chrome alloys.

Advantageously, the endoscopic scissor instrument of FIGS. 1-6 provides an improved automatic edge to edge preload of the opposed scissor blades while avoiding the problems associated with a bowed blade profile and biasing cams used in the prior art, and thus enables superior cutting quality for endoscopic scissor instruments where historically it has not been available.

In another aspect of the invention, the clevis of the endoscopic instrument of FIGS. 1 and 2 as described above can be configured to automatically provide a preload to the cutting edges of the two scissor blades as the scissor blades move past one another.

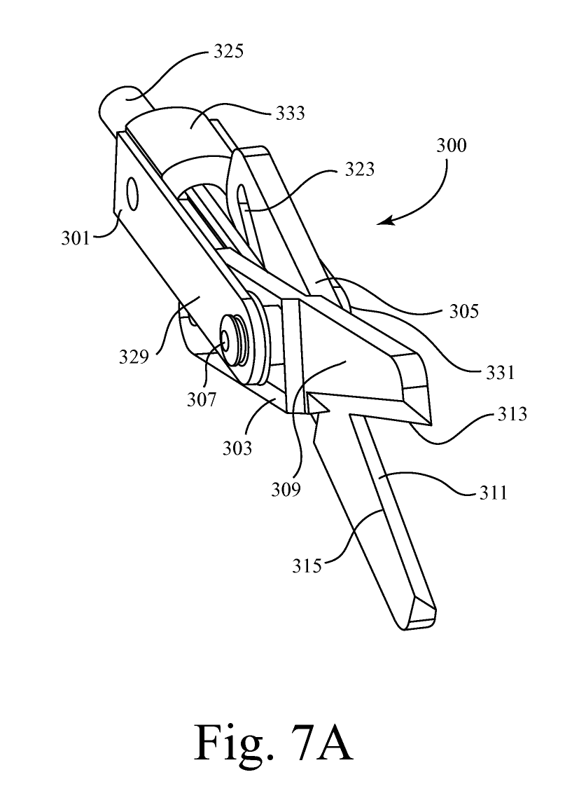

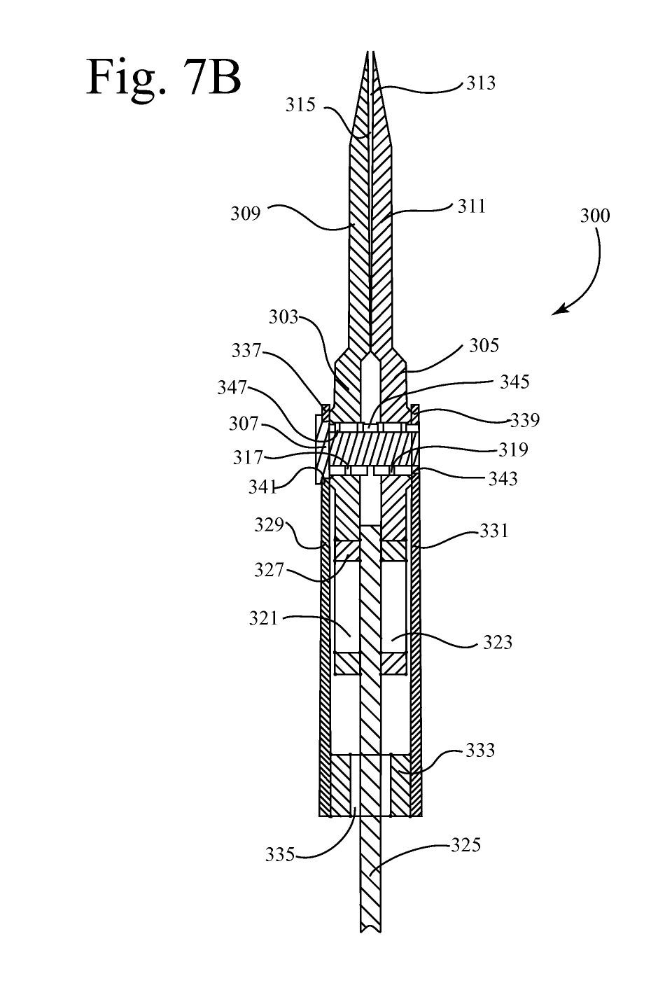

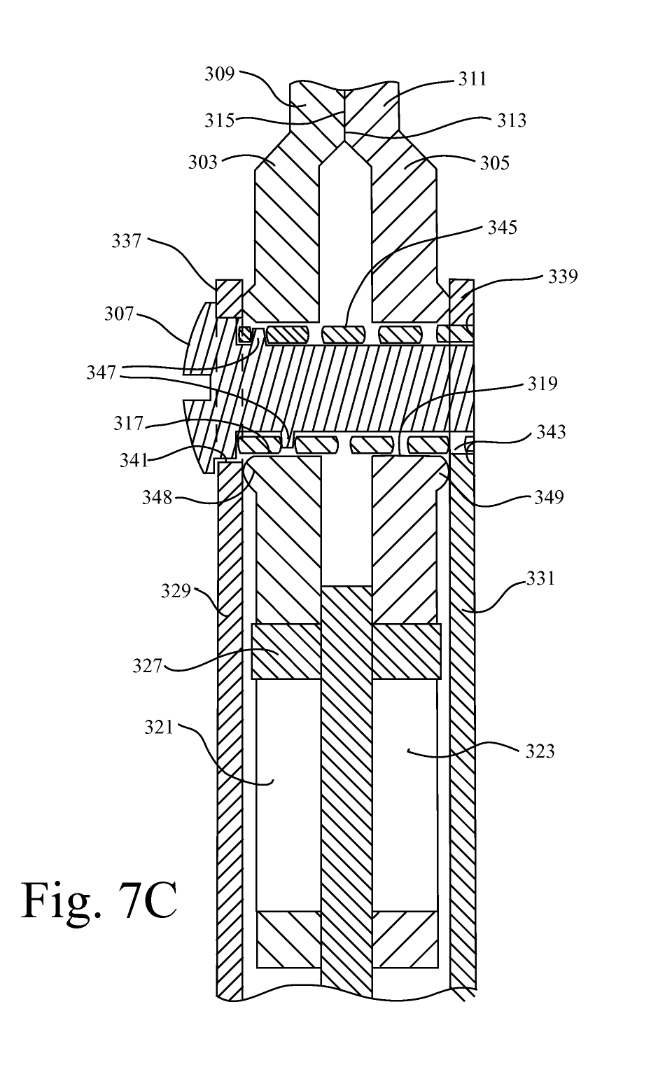

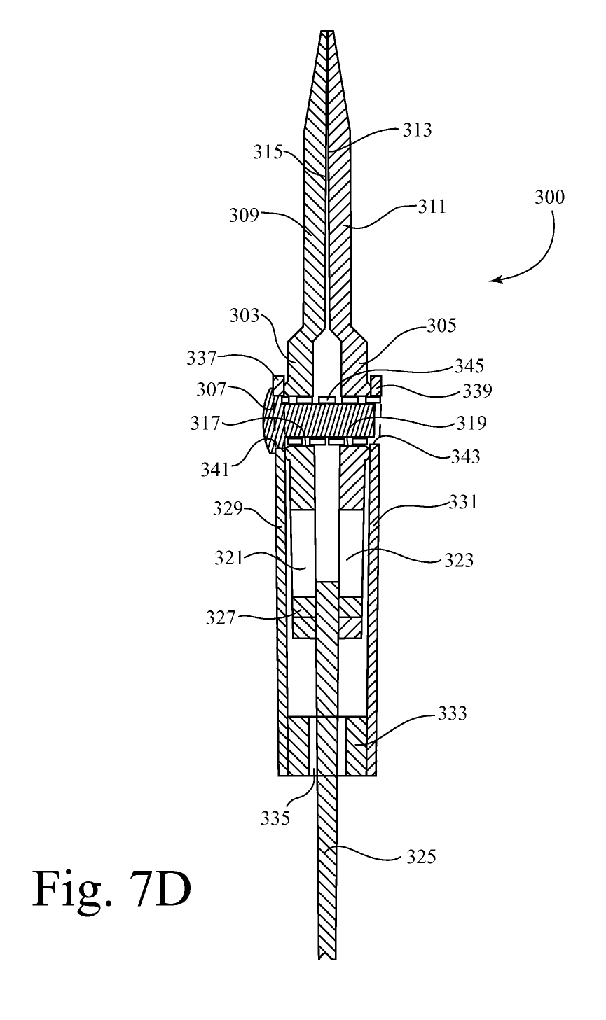

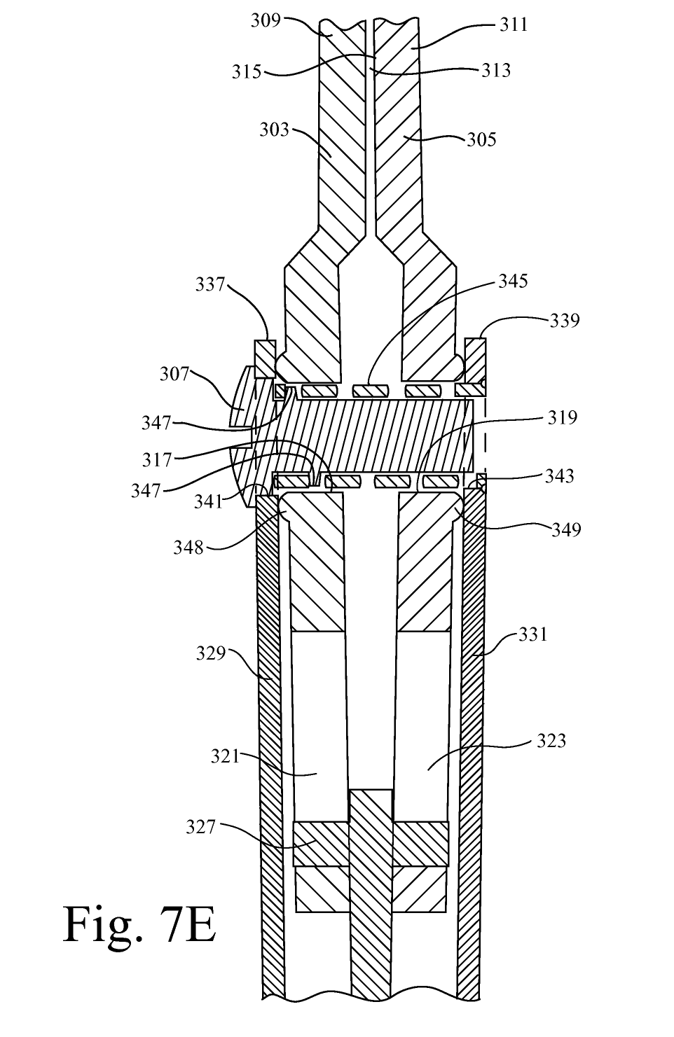

In an exemplary embodiment illustrated in FIGS. 7A-7E, the endoscopic instrument has an end-effector assembly 300 with a clevis 301 that is coupled to the distal end of a hollow tubular member (not shown). Scissor blades 303, 305 are rotatably mounted in the clevis 301 by a pivot screw 307. Each respective scissor blade 303, 305 has a distal longitudinally angled feature (309, 311) that defines a sharp cutting edge (313, 315) similar to the arrangement shown in FIG. 5C. As best shown in the cross-sectional views of FIGS. 7B and 7D, the angled features 309, 311 ensure that cutting edges 313, 315 are in intersecting planes as the scissor blades 303, 305 rotate relative to one another about the pivot screw 307 during operation.

As best shown in the cross-sectional views of FIGS. 7B-7E, the middle portion of each respective scissor blade 303, 305 defines a pivot hole (317, 319) that receives the body of the pivot screw 307. The proximal portion of each respective scissor blade (303, 305) defines a longitudinally extending cam-slot (321, 323) that is oriented at an oblique angle relative to the longitudinal axis of the assembly. The proximal portions of the two scissor blades 303, 305 are spaced apart from one another and receive the push rod actuator 325 therebetween. The actuator 325 includes a cam pin 327 that rides within the cam-slots 321, 323 of the respective scissor blades 303, 305 and effectuates rotational movement of the scissor blades relative to one another about the pivot screw 307. The cross-sectional view of FIG. 7B shows the open configuration of the scissor blades 303, 305. The cross-sectional view of FIG. 7D shows the fully-closed configuration of the scissor blades 303, 305.

The clevis 301 includes exterior leaf-spring arms 329, 331 that are disposed opposite one another and extend longitudinally with the scissor blade proximal portions disposed therebetween. The proximal ends of the leaf-spring arms 329, 331 are rigidly secured to a hub member 333. The hub member 333 has an internal channel 335 that provides for passage of the actuator 325 therethrough. The distal ends 337, 339 of the leaf-spring arms 329, 331 act as cantilever springs that resiliently deflect towards or away from one another under loading conditions brought about through the opening or closing of the scissors. The distal ends have respective thru-holes 341, 343 that are coaxial with the pivot holes 317, 319 of the scissor blades 303, 305 in order to receive the pivot screw 307 therethrough.

The body of pivot screw 307 is surrounded by a tension spring 345. One end of the tension spring 345 is welded to or otherwise secured to one of the leaf-spring arms (331). The other end of the tension spring 345 interfaces to radially-extending thread-like facets 347 of the pivot screw 307 as best shown in FIGS. 7C and 7E. The head of the pivot screw 307 interfaces to the other leaf spring arm (329). In this manner, the tension spring 345 is mechanically coupled between the opposed leaf-spring arms 329, 331. The pivot screw 307 can be manually turned such that the facets 347 slides along the length of the tension spring 345, and thus adjust the tension of the tension spring 345 as desired. The exterior surfaces of the respective scissor blades 303, 305 in the annular region surrounding the openings 317, 319 can be raised to form annular bumps 348, 349 that act like washers to minimize friction during rotational movement of the scissor blades 303, 305.

During rotational movement of the two scissor blades relative to one another about the pivot screw 307 (more particularly, during rotational movement from the open configuration (FIGS. 7B and 7C) to the fully-closed configuration (FIGS. 7D and 7E)), the angled profiles of the distal features 309, 311 of the opposed scissor blades 303, 305 causes the scissor blades 303, 305 to move away from one another in the transverse direction (i.e., the direction orthogonal to the longitudinal direction of the two blades). Such transverse movement is transmitted to the leaf-spring arms 329, 331 via the contact interface therebetween, resulting in deflection of the leaf-spring arms 329, 331 away from one another in the transverse direction. In response to such deflection, the leaf-spring arms 329, 331 as well as the tension spring 345 impart elastic forces that counteract the transverse movement of the scissor blades 303, 305 to ensure mating contact of the cutting edges 313, 315 of the opposed scissor blades. During rotational movement from the fully-closed configuration to the open configuration, the leaf-spring arms 329, 331 as well as the tension spring 345 impart elastic forces that cause transverse movement of the scissor blades 303, 305 toward one another to ensure mating contact of the cutting edges 313, 315 of the opposed scissor blades. In this manner, the leaf-spring arms 329, 331 and tension spring 345 provide spring moments that are primarily directed inward along the transverse direction. In the preferred embodiment, the elastic forces imparted by the leaf-spring arms 329, 345 and the tension spring 345 are constant during the full range of rotational movement of the scissor blades relative to one another, which maintains a consistent and continuous forceful contact of the cutting edges 313, 315 over the complete range of rotational movement of the scissor blades 303, 305.

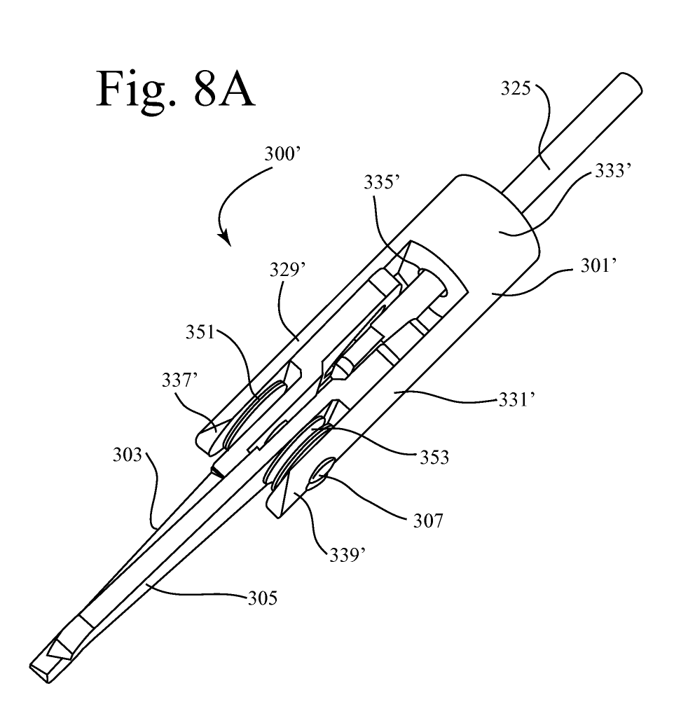

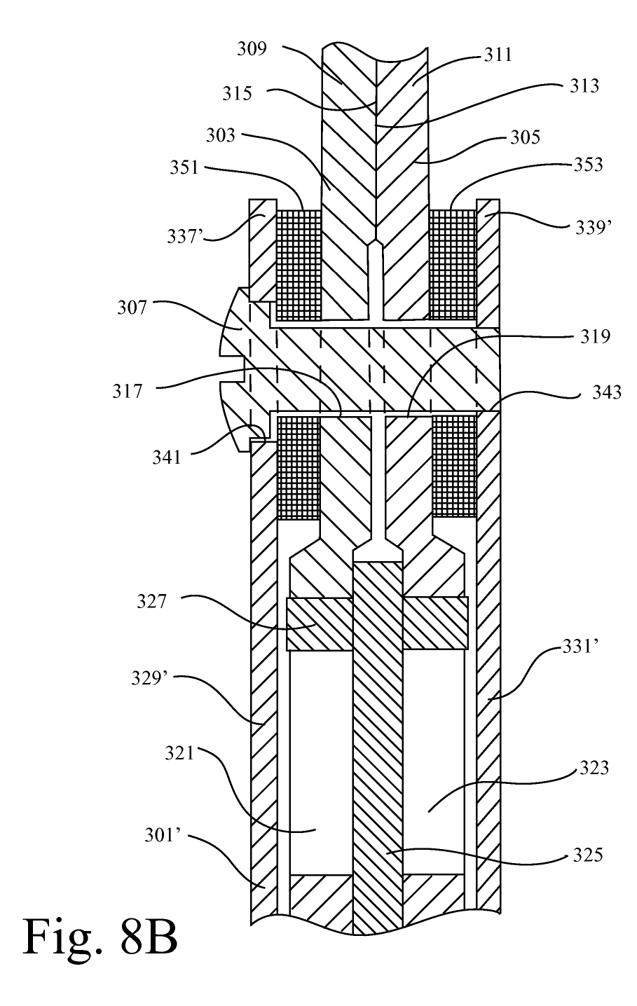

In another exemplary embodiment illustrated in FIGS. 8A and 8B, the endoscopic instrument has an end-effector assembly 300′ with a clevis 301′ that is coupled to the distal end of a hollow tubular member (not shown). Scissor blades 303, 305 are rotatably mounted in the clevis 301′ by a pivot screw 307. Each respective scissor blade 303, 305 has a distal longitudinally angled feature (309, 311) that defines a sharp cutting edge (313, 315) similar to the arrangement of FIGS. 7A to 7E. The angled features 309, 311 ensure that cutting edges 313, 315 are in intersecting planes as the scissor blades 303, 305 rotate relative to one another about the pivot screw 307 during operation.

As best shown in the cross-sectional view of FIG. 8B, the middle portion of each respective scissor blade 303, 305 defines an opening (317, 319) that receives the pivot screw 307 therethrough. The proximal portion of each respective scissor blade (303, 305) defines a longitudinally extending cam-slot (321, 323) that is oriented at an oblique angle relative to the longitudinal axis of the assembly. The proximal portions of the two scissor blades 303, 305 are spaced apart from one another and receive the push rod actuator 325 therebetween. The actuator 325 includes a cam pin 327 that rides within the cam-slots 321, 323 of the respective scissor blades 303, 305 and effectuates rotational movement of the scissor blades relative to one another about the pivot screw 307. The cross-sectional view of FIG. 8B shows the open configuration of the scissor blades 303, 305.

The clevis 301′ includes a hub 333′ with arms 329′, 331′ that extend distally therefrom. The arms 329′, 331′ are disposed opposite one another and extend longitudinally with the scissor blade proximal portions disposed therebetween. The hub member 333′ has an internal channel 335′ that provides for passage of the actuator 325 therethrough. The arms 329′, 331′ are substantially rigid in nature such that there is minimal deflection of the distal ends 337′, 339′ relative to one another under loading conditions. The distal ends 337′, 339′ have respective thru-holes 341, 343 that are coaxial with the openings 317, 319 of the scissor blades 303, 305 in order to receive the pivot screw 307 therethrough.

The body of the pivot screw 307 supports spring washers (in the exemplary embodiment, two spring washers 351, 353) disposed on opposite exterior sides of the scissor blades 303, 305 as shown in FIGS. 8A and 8B. In the preferred embodiment, the spring washers 351, 252 are Belleville-type spring washers. Spring washer 351 is disposed between the arm 329′ and blade 303. Spring washer 353 is disposed between the arm 331′ and blade 305. The head of the pivot screw 307 interfaces to clevis arm 329′. The end of the pivot screw 307 interfaces to clevis arm 331′ by a threaded interface or other suitable interface. In this manner, the spring washers 351, 353 are supported by the body of the pivot screw 307 intermediate the clevis arms 329′, 331′ and the central scissor blades 303, 305.

During rotational movement of the two scissor blades relative to one another about the pivot screw 307 (more particularly, during rotational movement from the open configuration to the fully-closed configuration), the angled profiles of the distal features 309, 311 of the opposed scissor blades 303, 305 causes the scissor blades 303, 305 to move away from one another in the transverse direction (i.e., the direction orthogonal to the longitudinal direction of the two blades). Such transverse movement is transmitted to the spring washers 351, 353 via the contact interface therebetween, resulting in compression of the spring washers 351, 353. In response to such compression, the spring washers 351, 353 impart elastic forces that counteract the transverse movement of the scissor blades 303, 305 to ensure mating contact of the cutting edges 313, 315 of the opposed scissor blades. In this manner, the spring washers 351, 353 provide spring moments that are primarily directed inward along the transverse direction. During rotational movement from the fully-closed configuration to the open configuration, the spring washers 351, 353 impart elastic forces that cause transverse movement of the scissor blades 303, 305 toward one another to ensure mating contact of the cutting edges 313, 315 of the opposed scissor blades. In the preferred embodiment, the elastic forces imparted by the spring washers 351, 353 are constant during the full range of rotational movement of the scissor blades relative to one another, which maintains a consistent and continuous forceful contact of the cutting edges 313, 315 over the complete range of rotational movement of the scissor blades 303, 305.

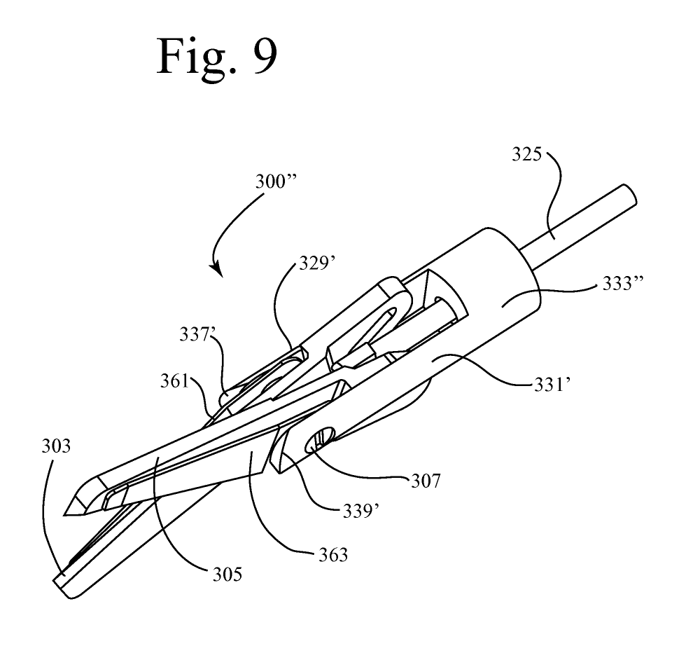

In yet another exemplary embodiment, the spring washers of the endoscopic scissors instrument of FIGS. 8A and 8B can be substituted with leaf springs 361, 363 as illustrated in FIG. 9. In this embodiment, the leaf springs 361, 363 are compressed during rotational movement of the scissor blades from the open to fully-closed configuration. In response to such compression, the leaf springs 361, 363 impart elastic forces that counteract the transverse movement of the scissor blades 303, 305 to ensure mating contact of the cutting edges 313, 315 of the opposed scissor blades. In this manner, the leaf springs 361, 363 provide spring moments that are primarily directed inward along the transverse direction. In the preferred embodiment, the elastic forces imparted by the leaf springs 361, 363 are constant during the full range of rotational movement of the scissor blades relative to one another, which maintains a consistent and continuous forceful contact of the cutting edges 313, 315 over the complete range of rotational movement of the scissor blades 303, 305. In the exemplary embodiment shown, the leaf springs 361, 363 have thru-holes that are coaxially aligned with the thru-holes of the first and second scissor blades and that receive the body of the pivot screw 307. The leaf springs 361, 363 extend distally from the respective distal ends 337′, 339′ of the clevis arms 329′, 331′ and longitudinally along a substantial portion of respective scissor blades 303, 305 as is evident from FIG. 9.

There have been described and illustrated herein scissors instruments with improved scissor blades. While particular embodiments of the invention have been described, it is not intended that the invention be limited thereto, as it is intended that the invention be as broad in scope as the art will allow and that the specification be read likewise. Thus, while the surgical scissors instrument illustrated herein for exemplary purposes were double acting scissors where both blades pivot relative to each other, it will be recognized that the invention can be applied to a single acting scissors with one blade fixed and the other blade pivoting relative to the fixed blade. It may also be applied to a scissors where only one blade incorporates the present invention coupled with a standard rigid opposing blade. Also, while particular actuation mechanisms were described for causing the rotation of the scissor blades, it will be appreciated that other mechanism could be utilized. Thus, for example, the instrument could be a flexible instrument with an outer tube formed from a coiled element which could be used through an endoscope channel or a rigid instrument with a relatively stiff outer tube of structural plastic or tubular metal which could be used through a laparoscope or arthroscope. In addition, while particular materials and dimensions have been disclosed for the scissor blades of the endoscopic scissors instruments, it will be understood that other materials and dimensions can be used. Moreover, while a particular unitary configuration of the respective scissor blades is shown, other non-unitary configurations can be used. For example, it is contemplated that the cutting features of the respective blades can be a separate and distinct part that is secured to the blade support of the scissor blade by welding (e.g., by laser welding, spot welding, resistance welding), one or more screws or rivets, or other suitable mechanical fixation means. In this configuration, the blade support can be realized from a wide range of materials, such as a stainless steel, plastics, ceramics, etc. It will therefore be appreciated by those skilled in the art that yet other modifications could be made to the provided invention without deviating from its spirit and scope as so claimed.

1-15. (canceled)

16. A surgical instrument comprising:
an elongate hollow member having a proximal end and a distal end;
an actuator that moves axially through said hollow member; and
an end effector assembly disposed adjacent said distal end of said elongate hollow member, said end effector assembly including a clevis, first and second scissor blades that are rotationally mounted in the clevis about a pivot mechanism, and coupling means that couples said actuator to at least one of said first and second scissor blades to provide for rotational movement of said first and second scissor blades with respect to one another in response to axial movement of said actuator;
wherein said first and second scissor blades each have a respective distal feature that defines a cutting edge, said distal features of said first and second scissor blades being longitudinally angled to ensure that said cutting edges are in intersection planes as the cutting edges contact one another during rotational movement of said first and second scissor blades relative to one another; and
wherein said end effector assembly includes spring bias means, disposed adjacent said pivot mechanism on at least one side of said first and second scissor blades, for biasing transverse movement of said scissor blades toward one another.

17. A surgical instrument according to claim 16, wherein:
said distal features of said first and second scissor blades are substantially rigid under loading conditions experienced during rotational movement of said first and second scissor blades relative to one another.

18. A surgical instrument according to claim 16, wherein:
said spring bias means generates a spring force acting on at least one of said first and second scissor blades such that an automatic preload force is imparted between the cutting edges of said first and second scissor blades.

19. A surgical instrument according to claim 18, wherein:
said spring force maintains a consistent and continuous mating contact of the opposed cutting edges of said first and second scissor blades.

20. A surgical instrument according to claim 19, wherein:
the spring force and the longitudinally angled distal features of said first and second scissor blades cooperate to maintain a continuous intersection of the opposed cutting edges of said first and second blades over the complete range of rotational movement of said first and second scissor blades.

21. A surgical instrument according to claim 16, wherein:
said spring bias means provides a spring moment that is primarily directed inward along the transverse direction.

22. A surgical instrument according to claim 16, wherein:
said first and second scissor blades have respective thru-holes coaxially aligned with one another, and said pivot mechanism comprises a body that is received by said thru-holes of said first and second scissor blades.

23. A surgical instrument according to claim 22, wherein:
said spring bias means comprises at least one leaf spring arm that is rigidly secured to a hub proximally disposed from said pivot mechanism, wherein said at least one leaf spring extends generally parallel to said longitudinal axis.

24. A surgical instrument according to claim 23, wherein:
said at least one leaf spring arm includes a thru-hole coaxially aligned with said thru-holes of said first and second scissor blades, wherein said thru-hole of said at least one leaf spring arm receives said body of said pivot mechanism.

25. A surgical instrument according to claim 24, wherein:
said spring bias means comprises two leaf spring arms that are rigidly secured to a hub proximally disposed from said pivot mechanism, wherein said two leaf spring arms extend generally parallel to said longitudinal axis on opposite external sides of said first and second scissor blades, said two leaf spring arms including respective thru-holes coaxially aligned with said thru-holes of said first and second scissor blades, wherein said thru-holes of said two leaf spring arms receives said body of said pivot mechanism.

26. A surgical instrument according to claim 23, wherein:
said spring bias means further comprises a tension spring that surrounds said body of said pivot mechanism.

27. A surgical instrument according to claim 26, wherein:
said pivot mechanism comprises a screw with at least one facet that extends radially from its body and engages said torsion spring, wherein said screw provides for manual adjustments of the spring tension forces afforded by said tension spring.

28. A surgical instrument according to claim 22, wherein:
said clevis includes a pivot support feature disposed on opposite exterior sides of said first and second scissor blades and supporting said pivot mechanism.

29. A surgical instrument according to claim 28, wherein:
said spring bias means comprises at least one spring washer that is coaxially aligned with said thru-holes of said first and second scissor blades, wherein said at least one spring washer receives said body of said pivot mechanism and is disposed between said pivot support feature and one of said first and second scissor blades.

30. A surgical instrument according to claim 28, wherein:
said spring bias means comprises two spring washers that are coaxially aligned with said thru-holes of said first and second scissor blades, wherein said two spring washers each receive said body of said pivot mechanism and are disposed on opposite external sides of said first and second scissor blades between said pivot support feature and one of said first and second scissor blades.

31. A surgical instrument according to claim 28, wherein:
said spring bias means comprises at least one leaf spring with a thru-hole that is coaxially aligned with said thru-holes of said first and second scissor blades, wherein said thru-hole of said leaf spring receives said body of said pivot mechanism, and wherein said leaf spring is disposed between said pivot support feature and one of said first and second scissor blades.

32. A surgical instrument according to claim 28, wherein:
said spring bias means comprises two leaf springs each with a thru-hole that is coaxially aligned with said thru-holes of said first and second scissor blades, wherein said thru-holes of said leaf springs receive said body of said pivot mechanism, and wherein said leaf springs are disposed on opposite external sides of said first and second scissor blades between said pivot support feature and one of said first and second scissor blades.

33. A surgical instrument according to claim 28, wherein:
said pivot support feature of said clevis comprises two arms that extend generally parallel to said longitudinal axis with distal portions of said first and second scissor blades disposed therebetween, each of said two arms including a respective thru-hole for receiving the body of the pivot mechanism therethrough.

34. A surgical instrument according to claim 16, wherein:
the cutting edges of the first and second scissor blades each have a length less than 50 mm.

35. A surgical instrument according to claim 16, wherein:
the cutting edges of the first and second scissor blades comprise sharpened cutting edges.

36. A surgical instrument according to claim 16, wherein:
said hollow member comprises a tube or coil that is flexible, rigid or plastically deformable.
